(19) United States
(12) Plant Patent Application Publication
(10) Pub. No.: US 2008/0256671 P1
(43) Pub. Date: Oct. 16, 2008

(54) Musa plant named '0714'

(76) Inventor: Issachar Ben-Zur, Tirat (IL)

Correspondence Address: PLANT HAVEN, INC., P.O. BOX 3056, SANTA BARBARA, CA, 93130, US

(21) Appl. No.: 11/981,887

(22) Filed: Nov. 2, 2007

(30) Foreign Application Priority Data
Apr. 10, 2007 (EP) 2007/0836

(51) Int. Cl. A01H 5/00

(52) U.S. Cl. PLT/156

(57) ABSTRACT

A new cultivar of Musa named ‘0714’ grown for its ornamental qualities, and whose leaf color is saturated burgundy-brown with uniformly distributed very small elongated green blotches. In combination these traits set ‘0714’ apart from all other existing varieties of Musa known to the inventor.

### GENUS

MUSA

### SPECIES

sumatrana

### DENOMINATION ‘0714’

### BACKGROUND OF THE INVENTION

The present invention relates to a new and distinct cultivar of Musa sumatrana, hereinafter referred to by the cultivar name, ‘0714’.

Musa sumatrana, whose common name is The Blood Banana or Red Banana Tree, is a native plant of Sumatra, Indonesia. Musa sumatrana can also be found described in the literature as Musa acuminata subsp. sumatrana or Musa acuminata subsp. zebrina. References to “sumatrana” as a cultivar name thus: ‘Sumatrana’, are erroneous: the plant is a native species or subspecies.

Musa sumatrana is noted for its elegant form consisting of broad glaucous green leaves with varying degrees of irregular “blotching” of burgundy-brown coloration. Juvenile plants of Musa sumatrana exhibit very little blotching, whereas mature plants may exhibit leaf blotching on half to two-thirds of the leaf surface area.

In August 2006, the inventor received a single plant of the species Musa sumatrana (unpatented). The inventor observed that this plant was distinct in that many of its leaves were unusually saturated with the burgundy-brown coloration, albeit with some green blotching. Although all of the leaves retained some small varying degrees of green, the whole plant appeared overall as burgundy-brown, rather than the reverse as is typical of the species.

The inventor, who is an experienced propagator of Musa by tissue culture, decided that the plant, though very attractive, was likely to be unstable in propagation and might revert over time to the green characteristic of its species. The inventor hoped that it would be possible to produce and isolate a new, distinct and stable clone by repeated cycles of tissue culture during which process all off-types would be discarded. Commencing in August 2006, at his laboratory in Tirat Yehuda, Israel, the inventor excised from the originally received plant the shoot tip of its pseudostem, containing the shoot meristem, and proceeded to establish new growth in vitro. Plants of Musa grow and proliferate (sucker) very rapidly under laboratory conditions and the inventor was able to carry out further propagations at approximately 4 week intervals. The resulting plants from each cycle were allowed to harden by growing at cooler temperature in order to increase the visibility of the burgundy-brown coloration and thereby to discard all plants which tended to grow as the species with predominantly green foliage. By February 2007, the inventor had completed four cycles of tissue culture with careful selection for the desired uniformity at each stage. After the fourth cycle, the inventor observed that all of the resulting plants were identical, with saturated burgundy-brown colored foliage which, upon close examination, exhibits uniformly distributed small green elongated blotches. This form, named ‘0714’ represents a new and distinct and stable variety of Musa sumatrana unlike any other varieties of Musa known to the inventor. The inventor has carried out further cycles of asexual reproduction, by tissue culture, both at his laboratory in Israel and under the inventor's supervision at a tissue culture laboratory in Bangkok, Thailand. The inventor has determined ‘0714’ is a new and stable variety of Musa which reproduces true to type in all its successive generations.

‘0714’ is derived from an unstable sport of the species Musa sumatrana, which is the parent variety and is the variety or species of Musa which most closely resembles ‘0714’. In comparison with Musa sumatrana’, ‘0714’ exhibits a burgundy-brown foliage color throughout, whereas Musa sumatrana’ is predominantly green with blotches of burgundy-brown.

This application claims the benefit of priority under 35 U.S.C. 119(f) of an application for European Community Plant Breeders Rights, Application No. 2007/0836, filed on Apr. 10, 2007.

### SUMMARY OF THE INVENTION

The following traits have been repeatedly observed and represent the distinguishing characteristics of the new Musa variety known as ‘0714’. These traits in combination distinguish ‘0714’ from all other existing varieties of Musa known to the inventor. ‘0714’ has not been tested under all possible conditions and phenotypic differences may be observed with variations in environmental, climatic, and cultural conditions, however, without any variance in genotype.
1. ‘0714’ is approximately 60 cm in height and 30 cm in width at six months of age from propagation.
2. The juvenile, unhardened, foliage of ‘0714’ is pale burgundy-brown is color, with pale green veins and pale green leaf edge surfaces. As growth proceeds and the plant hardens, these green regions become burgundy-brown in color.
3. The leaf color of a mature plant of ‘0714 is saturated burgundy-brown with occasional uniformly distributed very small elongated green blotches.

### DESCRIPTION OF THE DRAWINGS

The accompanying color drawings illustrate the overall appearance of the new Musa variety ‘0714’, showing the color as true as it is reasonably possible to obtain in colored reproductions of this type. Colors in the drawing may differ from the color values cited in the detailed botanical description, which accurately describe the actual colors of the new variety ‘0714’.

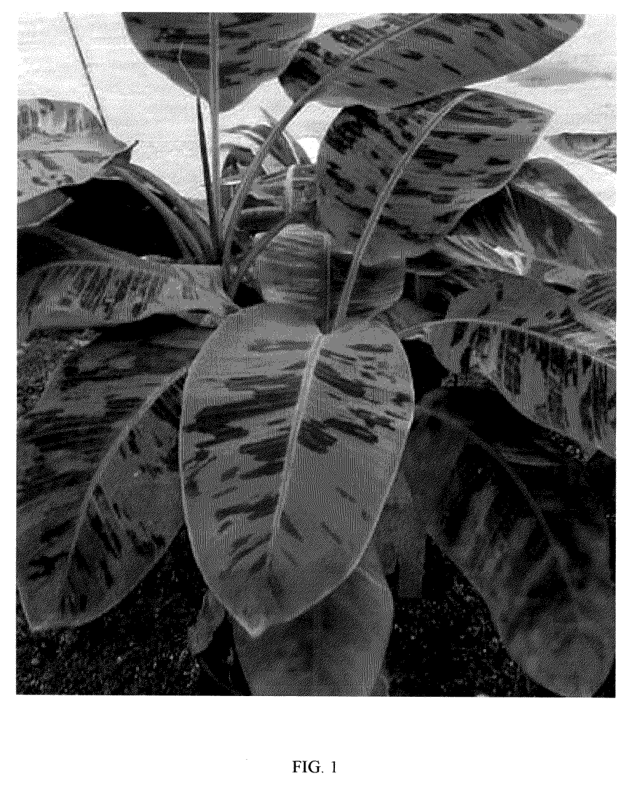

The drawing labeled as FIG. 1 illustrates a typical plant of Musa sumatrana and shows the predominant green coloration of the foliage together with the characteristic irregular burgundy-brown blotches.

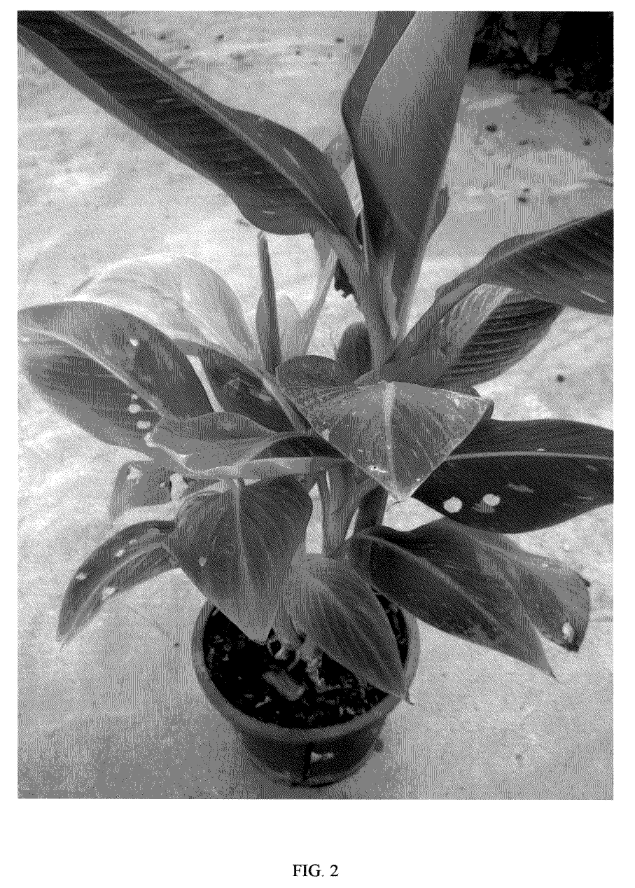

The drawing labeled as FIG.2 illustrates a plant in process of being stabilized by the inventor. The illustrated plant had been produced from the original discovered plant but was not considered sufficiently stable to be introduced.

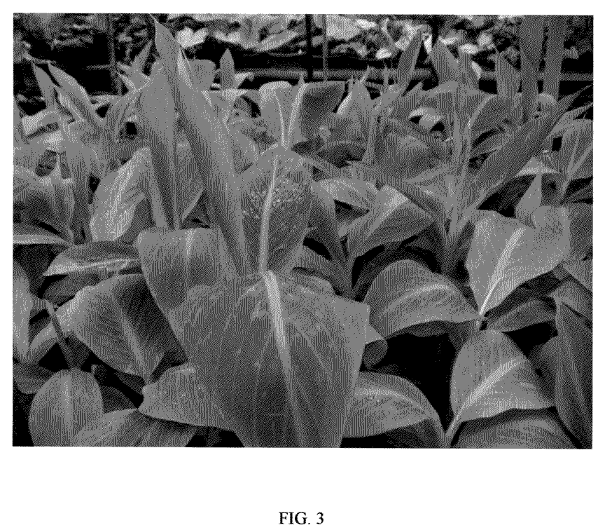

The drawing labeled as FIG.3 illustrates greenhouse-grown juvenile plants of ‘0714’ which resulted from the first cycle of propagation after the inventor determined that he had successfully stabilized the variety.

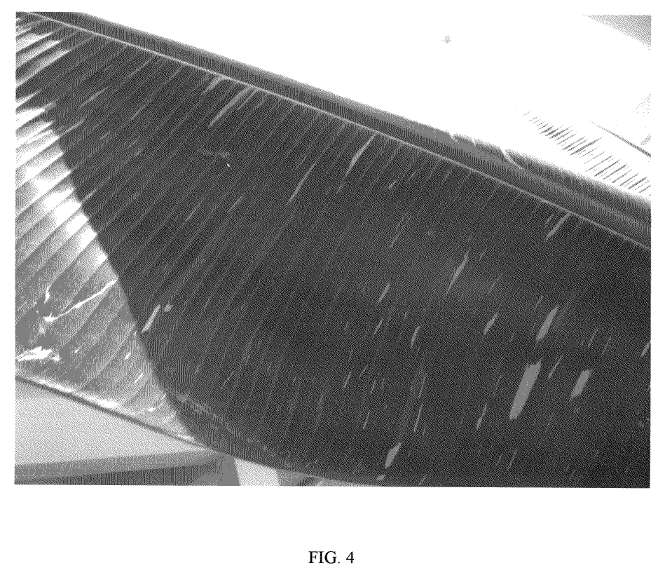

The drawing labeled as FIG.4 provides a close-up view of a typical section of a fully expanded leaf on a mature plant of ‘0714’. The leaf color is saturated burgundy-brown with occasional uniformly distributed very small elongated green blotches.

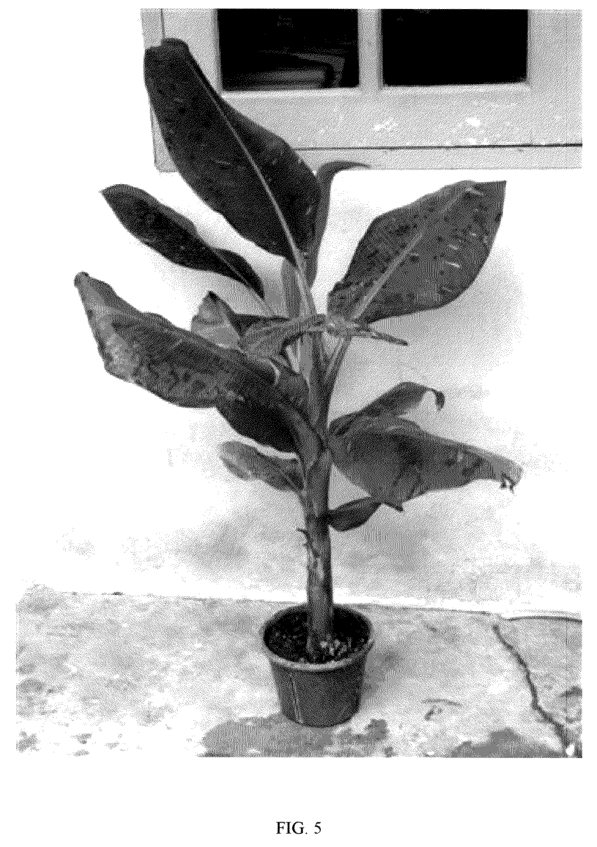

The drawing labeled as FIG. 5 illustrates a side view of a six months old plant of ‘0714’ growing out of doors in Bangkok, Thailand.

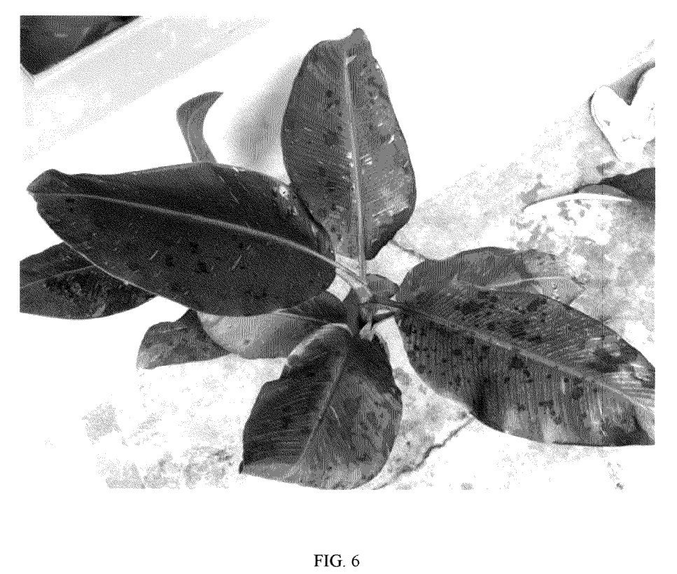

The drawing labeled as FIG. 6 illustrates a top view of the same plant illustrated in FIG. 5.

### BOTANICAL DESCRIPTION OF THE PLANT

The following is a detailed description of the new Musa cultivar named ‘0714’. Observations, measurements, color determinations, and comparisons were made in Bangkok Thailand, from six month old plants. Color determinations are in accordance with the 2001 Royal Horticultural Society Colour Chart from London, England, except where general color terms of ordinary dictionary significance are used. The growing requirements are similar to the species.
Botanical classification: Musa sumatrana ‘0714’.
Commercial classification: Ornamental foliage plant.
Species: Sumatrana
Common name: Banana, Blood Banana.
Use: Ornamental plant suitable as an indoor potted plant, landscape planting, and outdoor container plant.
Type: Tender perennial.
Parentage: Musa sumatrana.
Growth rate: Rapid in tissue culture propagation. When grown on in high temperature, high humidity conditions, seven expanded leaves are present after six months.
Growth habit: Upright, with arching leaves as growth proceeds.
Plant height: 60 cm.
Plant width: 40 cm.
Hardiness: USDA Zone 7b to 11
Propagation: Asexual propagation is accomplished using rhizome division and tissue culture.
Root system: Fleshy.
Pollination requirements: Not applicable
Cultural requirements: Plant in rich humus at a pH between 5.7 and 6.5. When grown out of doors, partial shade is recommended.
Crop time: 2-3 weeks to develop roots on an initial cutting at a recommended air temperature of 23-30 Centigrade, and approximately 5 months to reach commercial size.
Diseases and pests: Some susceptibility to spider mites. No serious disease problems known to the inventor.
Stem or trunk: Pseudostem comprised of unfurled leaf petioles.
   Shape.—Round, swollen at base.
   Size.—Length: 50-60 cm.
   Diameter.—20 mm towards base, tapering to 10 mm towards apex.
   Color.—(juvenile) Ranges from N144C to N144D (Mature) 178A to 179A.
Foliage:
   Type.—Evergreen.
   Leaf shape.—Wide lanceolate.
   Leaf length.—47 cm (base to apex, excluding petiole).
   Leaf width.—24 cm.
   Leaf arrangement.—whorled.
   Leaf apex.—lanceolate with a drip tip.
   Leaf base.—Rounded, asymmetric.
   Leaf division.—Simple.
   Leaf attachment.—petiolate sheath forming pseudostem.
   Petiole.—Petiole length (when detached from pseudostem): 10 cm-12 cm. Petiole diameter: 10 mm-15 mm Petiole color: Newest growth closest to N144C. Older foliage 179A. Petiole surface: Smooth, a little waxy.
   Leaf margins.—Entire.
   Leaf color (both surfaces, juvenile).—182C except veins and midrib.
   Leaf color (both surfaces, mature).—179A except for occasional very small elongated green blotches, 144C, of length 2 cm-3 cm, and width 1 mm-5 mm.
   Veins.—Pattern: Parallel. (Juvenile) Prominent, 3 mm in width, color N144C. (Mature) Appear only as shallow ridges, color as leaf, 179A.
   Leaf surface (both surfaces).—Glabrous, waxy.
   Leaf fragrance.—None observed.
   Stipules.—None observed.
Flower: No flowers have been observed.

1. A new and distinct variety of Musa plant named ‘0714’ as described and illustrated.
